(19) United States
(12) Patent Application Publication
(10) Pub. No.: US 2021/0268253 A1
(43) Pub. Date: Sep. 2, 2021

(54) CHAIN MAIL SURGICAL COLLAR AND METHOD OF PERCUTANEOUS DEVICE STABILIZATION THEREWITH

(71) Applicant: Cardiac Assist Holdings, LLC, Ann Arbor, MI (US)

(72) Inventor: Allen B. Kantrowitz, Ann Arbor, MI (US)

(73) Assignee: Cardiac Assist Holdings, LLC, Ann Arbor, MI (US)

(21) Appl. No.: 17/320,607

(22) Filed: May 14, 2021

Related U.S. Application Data
(63) Continuation-in-part of application No. PCT/US2019/061423, filed on Nov. 14, 2019.
Application No. PCT/US2019/061423 is a continuation-in-part of application No. PCT/US2020/060673, filed on Nov. 16, 2020.
(60) Provisional application No. 62/898,628, filed on Sep. 11, 2019.
(60) Provisional application No. 62/767,048, filed on Nov. 14, 2018.
(60) Provisional application No. 62/935,680, filed on Nov. 15, 2019.

(51) Int. Cl. A61M 39/02; A61F 2/00; A61M 1/00; A61F 13/00

(52) U.S. Cl. CPC A61M 39/0247; A61F 2/0063; A61M 60/117; A61F 13/00068; A61F 2002/0072; A61M 1/91

(57) ABSTRACT

Percutaneous access devices (PAD) or other implantable medical devices formed with chain mail are provided. The use of chain mail allows for a flexible PAD that promotes the formation of natural biologic seals between the skin and the device to form a barrier to microbial invasion into the body. Percutaneous access devices may be used for cardiac assist systems, peritoneal dialysis catheters, Steinman pin, Kirschner wires, chronic indwelling venous access catheters that require skin penetration, and osseo-integrated percutaneous medical appliances. Unlike conventional chain mail that is only formed in two dimensional sheets, chain mail is formed in elongated linear chains with occasional interlinks, two dimensional sheets, and in other configurations and combinations including three dimensional structures, pendant petals, elongated linear chains, combined fractal structures having a non-integer dimensionality intermediate between 1 and 3, and combinations thereof. Chain mail may be formed of combinations of simpler structures to form higher-order structures.

### RELATED APPLICATIONS

This application is a continuation-in-part of International Application No. PCT/US19/61423 filed 14 Nov. 2019, which in turn claims priority benefit of U.S. Provisional Application Ser. No. 62/767,048 filed 14 Nov. 2018 and 62/898,628 filed 11 Sep. 2019, and is also a continuation-in-part of International Application No. PCT/US20/60673 filed 16 Nov. 2020, which in turn claims priority benefit of U.S. Provisional Application Ser. No. 62/935,680 filed 15 Nov. 2019, the contents of the aforementioned priority documents are hereby incorporated by reference.

### FIELD OF THE INVENTION

The present invention in general relates to medical devices and systems and in particular to percutaneous access device (PAD) or other implantable medical devices formed with chain mail.

### BACKGROUND OF THE INVENTION

Heart disease is one of the leading causes of death. Currently, medical science cannot reverse the damage done to the cardiac muscle by heart disease. One solution for such patients is a heart transplant. However, the number of cardiac patients in need of a heart transplant far exceeds the limited supply of donor hearts available.

The scarcity of human hearts available for transplant, as well as the logistics necessary to undertake heart transplant surgery, makes an implantable cardiac assist device a viable option for many heart patients. A blood pump can be surgically implanted in, or adjacent to the cardiovascular system to augment the pumping action of the heart. The blood pump is sometimes referred to as a mechanical auxiliary ventricle assist device, dynamic aortic patch, balloon pump, mechanical circulatory assist device, or a total mechanical heart. Alternatively, the blood pump can be inserted endovascularly.

Typically, the blood pump systems include a driveline that serves as a power and/or signal conduit between the blood pump internal to the patient and a controller/console external to the patient.

Often a percutaneous access device (PAD) can be surgically implanted in the body at the location in the skin where the driveline penetrates the skin to provide a through-the-skin coupling for connecting the supply tube to an extra-corporeal fluid pressure source. Alternatively, the fluid pressure source can be implanted wholly within the body, energized by electromagnetic means across intact skin, or energized by or chemical energy found within the body or some other means. Electrical leads from electrodes implanted in the myocardium are likewise brought out through the skin by means of the PAD. The aortic valve status or any cardiovascular parameter that is associated with this status can be employed to control the fluid pressure source to inflate and deflate the inflatable chamber in a predetermined synchronous relationship with the heart action.

The surface of the driveline, or of the optional PAD used in cardiac assist systems may have characteristics which promote the formation of a natural biologic seal between the skin and the device to form a barrier to microbial invasion into the body at the skin penetration site. Percutaneous access devices may also illustratively be used for other devices including peritoneal dialysis catheters, Steinman pin, Kirschner wires, and chronic indwelling venous access catheters that require skin penetration. More generally, medical appliances which are implanted so as to cross the skin surface and therefore violate the “barrier function” of the skin, may also illustratively be used for other medical purposes including peritoneal dialysis catheters and, chronic indwelling venous access catheters, neurologic prostheses, osseointegrated prostheses, drug pumps, and other treatments that require skin penetration.

FIG. 1 illustrates wearable and implanted components of an exemplary prior art cardiac assist system. A PAD 10 serves as an attachment point for an external supply line 12 that supplies air or fluid from a wearable external drive unit (EDU) 14. The EDU 14 is powered by a wearable battery pack 16. Inside the body of the patient, a drive line 18 is attached to the PAD 10 and provides an air or fluid conduit to a cardiac assist device 20.

A common problem associated with implantation of a PAD or other skin penetrating appliance is skin regeneration about the periphery of the appliance to form an immunoprotective seal against infection. New cell growth and maintenance is typically frustrated by the considerable mechanical forces exerted on the interfacial layer of cells. In order to facilitate skin regeneration about the exterior of the appliance, subject cells are often harvested and grown in culture onto appliance surfaces for several days prior to implantation in order to allow an interfacial cell layer to colonize appliance surfaces in advance of implantation. Unfortunately, cell culturing has met with limited acceptance owing to the need for a cell harvesting surgical procedure preceding the implantation procedure. Additionally, maintaining tissue culture integrity is also a complex and time-consuming task.

A related context in which cell growth is needed is wound healing, with DACRON® based random felt meshes have been used to promote cell regrowth in the vicinity of a wound, or to promote tissue anchorage by fibrous-scar-investment of a medical device crossing the skin surface. Such felts have uncontrolled pore sizes, some of which function as safe-haven microenviornments that harbor bacterial growth.

U.S. Pat. No. 7,704,225 to Kantrowitz solves many of these aforementioned problems by providing cell channeling contours, porous biodegradable polymers and the application of vacuum to promote cellular growth towards the surface the neck of a PAD. The facilitating of rapid cellular colonization of a PAD neck allows the subject to act as their own cell culture facility and as such affords more rapid stabilization of the PAD, and lower incidence of wound separation and infection.

FIG. 2 depicts a PAD generally at 100 as shown in U.S. Pat. No. 10,258,784 to Kantrowitz. A cap 102 is formed of a material such as silicone, a polymer or a metal and serves to keep debris from entering the device 100. Preferably, the cap 102 is remote from the surface of the epidermis E. The medical appliance 34 depicted as a catheter and vacuum or hydrodynamic draw tubing 104 pass through complementary openings 106 and 108, respectively formed in the cap 102. The tubing 104 provides fluid communication between a vacuum or hydrodynamic draw source 22 and an inner sleeve 13. The inner sleeve 13 is characterized by a large and rigid pore matrix 19 in fluid communication to a vacuum source 22 such that the source 22 draws (arrow 22D) tissue fluid and fibroblasts 21 into the sleeve 13 Sleeve 13 has a surface 24 that is optionally nanotextured to promote fibroblast adhesion. The surface 24 is optionally decorated with a pattern of contoured cell-conveying channels. It is appreciated that inner sleeve 13 optionally includes matrix 26 thereover, a coating substance 27, or a combination thereof. The coating 27 is appreciated to need not cover the entire surface 24. The tissue contacting surface 29 of substance 27 is optionally nanotextured. A flange 112 is provided to stabilize the implanted device 100 within the subcuteanous layer S. The flange 112 is constructed from materials and formed by methods conventional to the art. For example, those detailed in U.S. Pat. Nos. 4,634,422; 4,668,222; 5,059,186; 5,120,313; 5,250,025; 5,814,058; 5,997,524; and 6,503,228.

FIGS. 3A-3C illustrate a modular external interface housing 200 coupled to the PAD 100 as disclosed in U.S. Application No. 15/555,952 to Subilski. The modular external interface 200 forms a collar about the neck 110 of the PAD 100 with the main body 216 with a locking feature 218, such as a male extension that engages a female receptacle or cavity as a mechanical overlap connection. In a specific embodiment the main body 216 is made of silicone. The collar seal between the main body 216 and the neck 110 of the PAD 100 forms a hermetic seal with a gasket 230, which in a specific embodiment is a flexible gasket integrated into the main body 216. In a specific embodiment the gasket 230 may be a floating gasket. The stabilization of the PAD 100 within the skin to form a germ-free barrier requires subject cells to grow onto the neck surfaces 17 as shown in FIG. 2 of the PAD 100 adjacent to the subject's epidermis E. The neck surface region 17 is adapted to promote growth of autologous fibroblast cells thereon. A suitable exterior side surface substrate for fibroblast growth is a nanotextured polycarbonate (LEXAN®). The modular external interface 200 has a central opening adapted for at least one drive line 220 for insertion into a PAD, and a portal 224 for a vacuum line 222.

The modular external interface 200 is secured and sealed to an outer layer of a patient's skin with a medical dressing. In a specific embodiment the medical dressing is a preform patterned and shaped to conform to the exterior of the modular external interface 200. In a specific embodiment the medical dressing preform may be in two halves (212 214) that overlap. In a specific embodiment the medical dressing preform may be transparent. In a specific embodiment the medical dressing preform may be made of Tegaderm™ manufactured by Minnesota Mining and Manufacturing Company.

Despite the advances in PAD design and the securement of PAD to a subject's skin there continues to be a problem of disrupting the formation and maintaining of skin layers about the PAD with respect to flexible or pliable drivelines during the healing process. Infection at the site of PAD used with pliable and flexible drivelines continues to occur as the seal between the layers of skin and the bendable driveline tends to either not fully form or fails as the driveline flexes at the insertion site.

There is a continuing need for improved percutaneous access devices that minimize the disruptive forces to nascent layers of skin that are being formed during the healing process, as well as maintaining an infection preventive seal around flexible or pliable drivelines

### SUMMARY OF THE INVENTION

A percutaneous access device (PAD) is provided that includes a chain mail collar formed from a plurality of intersecting ringlets formed of biocompatible materials, and a central aperture through the chain mail collar. The device may further include a sensor for measuring healing in vivo proximal to the chain mail collar. The plurality of intersecting ringlets may have a shape of planar circular, planar triangular, planar rectilinear, planar square, planar pentagonal, planar oval, planar hexagonal, three dimensionally kinked rectilinear, and three dimensionally kinked oval. The chain mail collar may be formed by three dimensional (3-D) printing.

A process is provided of repairing a hernia that includes inserting a chain mail sheet formed from a plurality of intersecting ringlets formed of biocompatible materials proximal to weakened or ruptured luminal area of a subject, and adhering the chain mail sheet as a reinforcement mesh across the weakened or ruptured luminal area. The chain mail sheet may be inserted laparoscopically and is collapsed for insertion into the patient and then unfurled at the weakened or ruptured luminal area. The process further includes creating a vacuum draw or hydrostatic draw through the chain mail sheet.

### BRIEF DESCRIPTION OF THE DRAWINGS

The subject matter that is regarded as the invention is particularly pointed out and distinctly claimed in the claims at the conclusion of the specification. The foregoing and other objects, features, and advantages of the invention are apparent from the following detailed description taken in conjunction with the accompanying drawings in which like reference numerals refer to like parts throughout the several views, and wherein:

FIG. 1 illustrates prior art wearable and implanted components of a cardiac assist system with a percutaneous access device (PAD) and internal driveline;

FIG. 2 is a prior art, partial cutaway view of a flanged percutaneous access device (PAD) with relative dimensions of aspect exaggerated for visual clarity;

FIGS. 3A-3C are perspective views of a prior art modular external interface seal for a PAD appliance secured with adhesive dressings to a subject;

FIG. 4A illustrates an existing sheet of chain mail material;

FIG. 4B illustrates a close up view of FIG. 4A showing the linked ringlets that form the chain mail material;

FIGS. 5 illustrates an elongated chain mail collar attached to a flexible or pliable driveline at an insertion site in accordance with embodiments of the invention;

FIGS. 6A-6F are a series of side views of percutaneous access devices formed of chain mail in accordance with an embodiment of the invention;

FIGS. 7A-7H illustrate the variety of shapes that a ringlet may be formed as in accordance with embodiments of the invention; and

FIG. 8 illustrates a system for suppling intravenous fluids and a vacuum via an integrated muti-lumen tubes to the modular external interface seal of FIGS. 3A-3C for PAD appliances equipped with a chain mail collar, environmental sensors, an air filter, and a viewing window in accordance with embodiments of the invention.

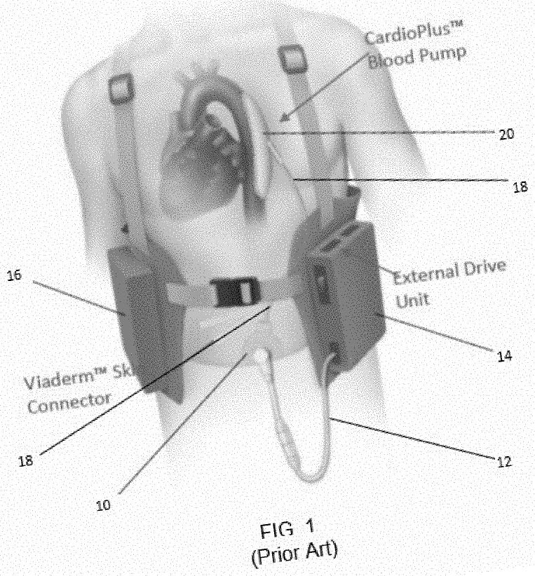

### DETAILED DESCRIPTION OF THE INVENTION

Embodiments of the invention provide percutaneous access devices (PAD) or other implantable medical devices formed with chain mail. The use of chain mail allows for a flexible PAD as used herein may include PAD used in cardiac assist systems that promote the formation of a natural biologic seal between the skin and the device to form a barrier to microbial invasion into the body. Percutaneous access devices may also illustratively be used for other devices including peritoneal dialysis catheters, Steinman pin, Kirschner wires, chronic indwelling venous access catheters that require skin penetration, and osseo-integrated percutaneous medical appliances.

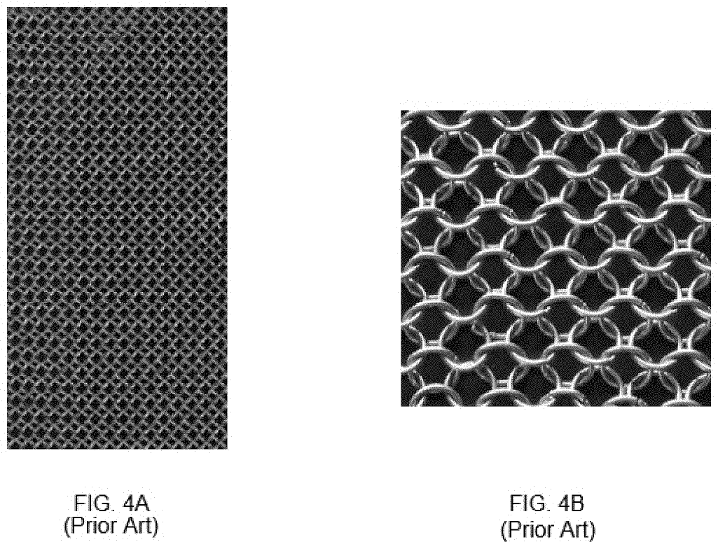

Chain mail refers to sheets of material that are formed from ringlets linked together in a pattern. Unlike conventional chain mail that is only formed in two dimensional sheets, according to the present invention, chain mail is formed in elongated linear chains with occasional interlinks, two dimensional sheets, and in other configurations and combinations including three dimensional structures, pendant petals, elongated linear chains, combined fractal structures having a non-integer dimensionality intermediate between 1 and 3, and combinations thereof. Chain mail can also be formed of combinations of simpler structures to form higher-order structures. FIG. 4A illustrates a typical sheet of chain mail. FIG. 4B illustrates a close up view of FIG. 4A that more clearly shows the interlocked ringlets that form the chain mail sheet. Chain mail according to the present invention, provides strength and flexibility to deform under the movement associated with a percutaneous implant or an implant adherent or otherwise mechanically coupled to any flexible, deformable, body tissue, all while providing infiltration volume for fibroblasts, infiltration and hydrostatic or mechanical vacuum draw through the ringlets to bring the well-known advantages of negative-pressure-wound therapy to the site of wound healing such as reducing bioburden and stimulating local fibroblast proliferation and migration. In some inventive embodiments, the ringlets are sized to define a central opening sized to accommodate fibroblast growth therein so as to form an extended scaffold for fibroblast stabilization of the chain mail. Moreover, the interstices of the chain mail structure can be interwoven, crocheted, knitted or otherwise threaded with fibers, illustratively including monofilament, polyfilament, hollow fibers, and combinations thereof; which supplement the chainmail structure in various ways to provide improved functionality such as increased tensile strength with biodegradable fibers, increased tensile strength with non-biodegradable fibers, physiologic sensors, fiber optic illumination for sensing, physiologic monitoring, or facilitating optically dependent chemical reactions, electrical wires for sensing, physiologic monitoring, or facilitating electrically dependent chemical reactions, and hydrostatic or mechanical vacuum draw through the ringlets to bring the well-known advantages of negative-pres sure-wound therapy to the site of wound healing such as reducing bioburden and stimulating local fibroblast proliferation and migration. Such interwoven fibers may extend beyond the edges of the chain mail to serve as extended anchorages to the adjacent tissues and may terminate in an optionally detachable surgical needle which can be advanced into adjacent tissues or may terminate in another fixture which can engage surgically, endoscopically, percutaneously, or otherwise secured to anchor devices in the adjacent tissues. Similarly, antimicrobials or sensors can be fed into the interstitial network created by the ringlets of the chain mail. Similarly, the interstices can be used to deliver biologically active chemicals such as fibroblast growth factors and the like. It is recognized and preferred that infiltration of the chain mail and investment of the chain mail with fibroblasts, collagen fibers and other biologic tissues will serve to increase the mechanical compliance of the chain mail in mechanical deformation modes such as tension, flexion, torsion, and compression.

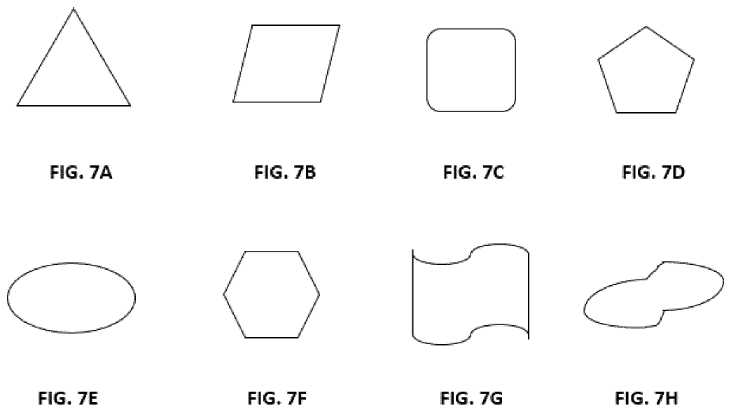

A ringlet is readily formed in a variety of shapes besides the simple circular ringlet per FIGS. 4A and 4B, that illustratively include planar triangular (FIG. 7A), planar rectilinear (FIG. 7B), planar square (FIG. 7C), planar pentagonal (FIG. 7D), planar oval (FIG. 7E), planar hexagonal (FIG. 7F), three dimensionally kinked rectilinear (FIG. 7G), and three dimensionally kinked oval (FIG. 7H). In some inventive embodiments, the ringlets are sized to define a central opening sized to accommodate fibroblast growth therein so as to form an extended scaffold for fibroblast stabilization of the chain mail. As a result, the degree of connectivity, the number of contiguous ringlets linked to a given ringlet, is adjusted to create a desired degree of open area and rigidity. Typical degrees of connectivity range from 2 to 20, depending on the shape of the ringlets and the dimensionality of the chain mail. It is appreciated that different shaped ringlets are combined to form a chain mail collar.

Ringlets are formed of a variety of biocompatible materials including those that are bio-retained as well as those that are biodegradable, and a combination thereof. Materials from which a ringlet are formed illustratively include titanium, alloys containing a majority by weight titanium, tungsten, and tantalum; stainless steel; polyurethane, fluorpolymers, perfluoropolymers, silicones, polylactides, biodegradable polymers, and non-biodegradable polymers. It is appreciated that a ringlet is readily provided with a surface coating or treatment. Surface coatings operative herein include any of the aforementioned polymeric materials from which ringlets are formed, antimicrobials, fibroblast adhesion promoters, fibroblast stimulation promoters, vascular growth factors, bioactive growth factors and a combination thereof. Surface treatments operative in the present invention include anodization of ringlets formed of metal, plasma surface roughening, chemical surface roughening, carbidization or anodization of ringlets formed of metal, and mechanical polishing. It is further appreciated that a facing layer of tiles can be added to thereto in a miniature form of that contemplated to protect spacecraft. https://newatlas.com/3d-print-space-fabric/49105. The facing tiles formed of any of the aforementioned substances and can be in the form of tiles, polymeric sheets, fabrics or combinations thereof.

In some inventive embodiments, chain mail of the present invention is formed by three dimensional printing. An exemplary processes of 3D printing with implant compatible metals is detailed in L. E. Murr et al. J. of Matls. Res.& Tech.; 2012, 1(1), 42-54; while such a process for biocompatible polymers is detailed in Q. Chen et al., ACS Appl. Mater. Interfaces 2017, 9(4), 4015-4023.

Embodiments of the inventive percutaneous access devices (PAD) or other implantable medical devices formed with chain mail may have ringlets formed of materials and metals suitable for medical use illustratively including titanium, tungsten, tantalum, or alloys in which any one of the aforementioned metals constitute the atomic percent majority of the alloy; and stainless steel. The individual ringlets may have diameters that are sized to favor attachment to the skin via fibroblast attachment. The additional layers of chain mail may be added to specific areas of a sheet of chain mail to provide additional reinforcement or to add three dimensional (3-D) features such as a lip that in some embodiments functions as an anchoring extension. The additional layers of chain mail may be joined to underlying layers of chain mail via entanglement of ringlets, spot welds, stitching, or adhesives. It is appreciated that 3-D printing machines may be used to form multidimensional shapes using chain mail patterns in the layers as a two-dimensional (2-D) sheet is extended to a 3-D sheet of chain mail. The chain mail may be treated with anti-microbial substances and substances that encourage fibroblast attachment and growth on the chain mail.

Embodiments of the chain mail may be made into collapsible forms that may then be inserted into a patient and then deployed or unfurled at a target site to minimize a required incision during a surgical procedure, such as through a laparoscopic or other minimally invasive medical procedure. Embodiments of chain mail mesh sheets may be used for hernia repairs as a reinforcement mesh in a weakened or ruptured area.

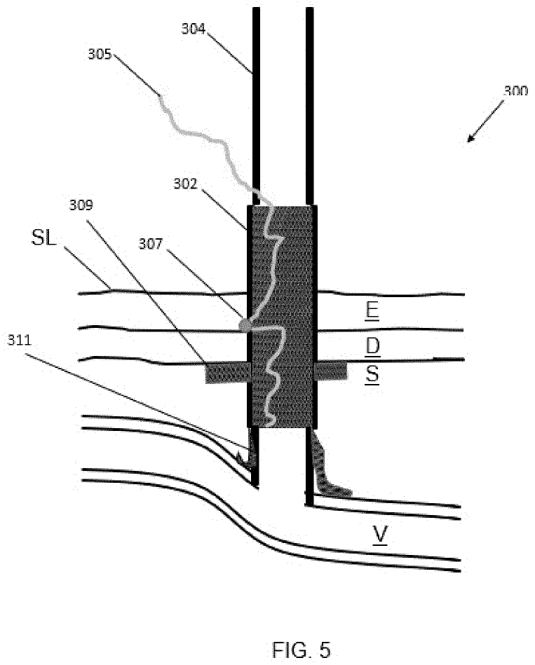

Referring now to the figures, FIG. 5 illustrates an installation 300 of an elongated chain mail collar 302 attached to a flexible or pliable driveline 304 at an insertion site. The chain mail collar 302 is percutaneous through the skin line (SL) and the epidermis, dermis, and subcutaneous layers that are denoted at E, D, and S, respectively, and the driveline extends to-a vein V. The chain mail collar in some inventive embodiments is adhered to the driveline 304 with an adhesive. In a specific inventive embodiment, the chain mail collar 302 may be attached to the driveline 304 by sutures. In a specific embodiment the chain mail collar 302 may be locally heated to a temperature sufficient to melt an outer surface of the driveline 304 to the chain mail collar 302. In still other inventive embodiments, a fiber 305 is interwoven into the collar 302. Only a single fiber 305 is shown for visual clarity, it is appreciated that numerous such fibers can be present. The fiber 305 illustratively including monofilament, polyfilament, hollow fibers, or combinations thereof. A fiber 305 modifies properties by affording capillary draw to promote drying, fibroblast infiltration, and in some circumstances monitoring of serous fluid for early indications of infection. Hollow fibers are particularly well suited for such sampling. The chain mail collar 302 in some embodiments includes transverse appendages 309 of mail. The mail of the appendage 309 forming petals, an annulus, linear chain extensions, or combinations thereof. The chain mail collar 302 in still other embodiments includes distal appendages 311 of mail forming petals, an annulus, linear chain extensions, or combinations thereof.

In some embodiments, a sensor 307 is provided either alone or in combination with a fiber 305. A sensor 307 illustratively including a thermocouple; a gas sensor such as oxygen, or sulfur; exudate biochemical such as electrolytes such as sodium, potassium, or chloride; small molecules such as urea, creatinine, fibrinogen, matrix metalloproteinases (MMPs); proteins such as tumour necrosis factor (TNFa) and C-reactive protein (CRP); and combinations thereof. The sensor 307 having leads extending external to the skin such as via a fiber 305 or monitored wirelessly.

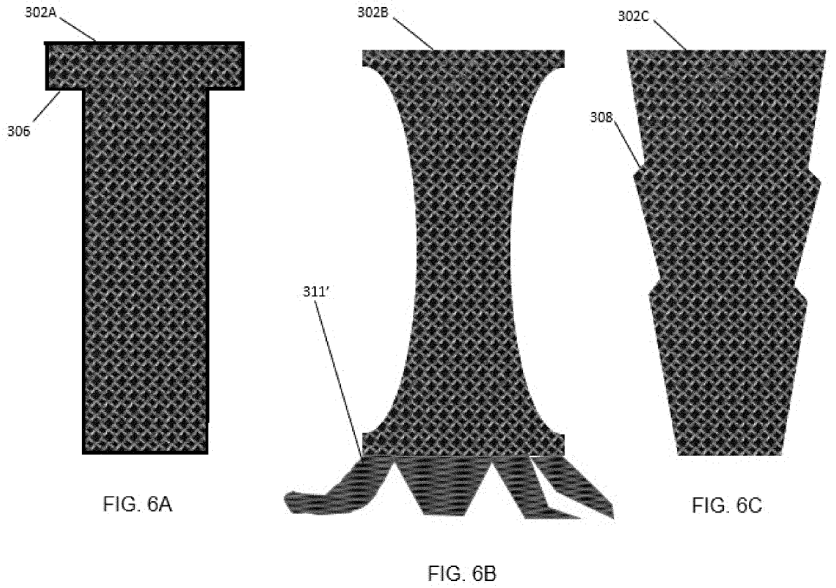

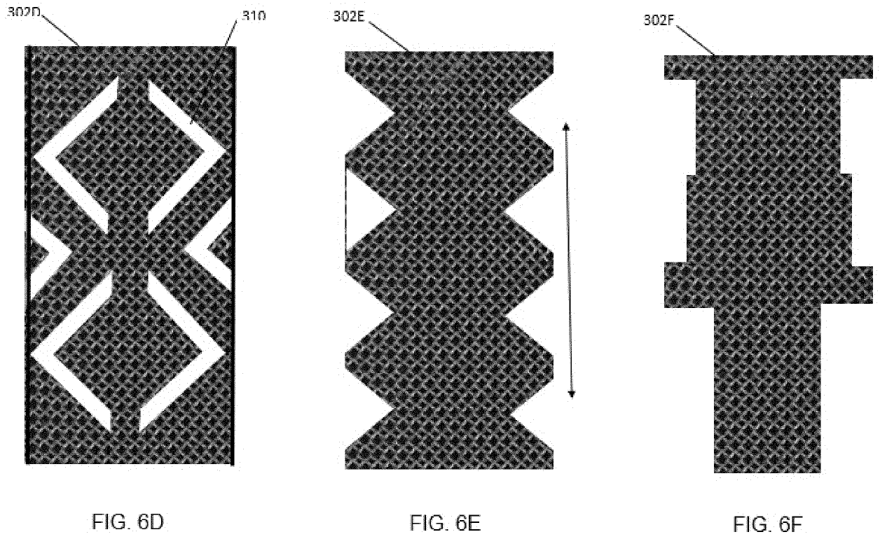

FIGS. 6A-6F are a series of side views of percutaneous access devices (PAD) formed of chain mail in accordance with an embodiment of the invention. FIG. 6A shows a PAD with an upper lip 306 that extends outward around the circumference. The lip 306 may be formed as described above. FIG. 6B the chain mail formed PAD 302B has a tapered shape having distal appendages 311′. In FIG. 6C the chain mail formed PAD 302C has a series of serrated steps 308 to help anchor the PAD 302C to the skin layers at an insertion site. In FIG. 6D the chain mail formed PAD 302D has cutouts 310 that allow the PAD 302D to be folded origami style for insertion into a surgical site, where the PAD 302D may then be unfolded to the expanded size of the PAD 302D. In FIG. 6E the chain mail formed PAD 302E is collapsible, and that allows the PAD 302E to be in a collapsed form for insertion into a surgical site, where the PAD 302E may then be expanded in size. In FIG. 6F the chain mail formed PAD 302E has varying regions of thicknesses of the chain mail.

An inventive chain mail collar is connected, in some inventive embodiments, to a vacuum source. A vacuum source may be any source operable for creating negative pressure in or around the device. A vacuum source illustratively includes a passive vacuum such as a vacuum tube or bottle, or an active vacuum source illustratively a mechanical pump, a syringe, or other vacuum source. A vacuum source optionally applies a continuous or intermittent negative pressure. The magnitude of the negative pressure is optionally adjustable, constant, or variable. In some embodiments an intermittent vacuum is used. Alternatively, a hydrodynamic draw agent is provided that draws fluid from the tissue surrounding along the chain mail ringlets or fibers woven therethrough. Without intending to be bound by a particular theory, capillary draw is believed to be operative in drawing exudate in a direction of the vacuum drawn to promote healing and stabilization of the chain mail collar. A hydrodynamic draw source illustratively includes a super absorbent polymer such as sodium polyacrylate, polyacrylamide copolymer, ethylene maleic anhydride copolymer, cross-linked carboxymethylcellulose, polyvinyl alcohol copolymers, cross-linked polyethylene oxide, and starch grafted copolymer of polyacrylonitrile; high osmotic pressure compositions, such as water soluble salts; and capillary flow draw agents such as dry silica, or other dry hydrophilic powders such as cellulosic material.

It is increasingly common for catheters and percutaneous access such as peripherally inserted central catheters (PICC), skeletal guide wires, cardiac assist device lines, or other instruments to be kept in place for weeks or months. The increased time in which such devices are maintained across the skin increases the likelihood of instrument related infection. Another common implantable device that breaks the skin and may be a source of infection are blood pumps that may be surgically implanted in, or adjacent to the cardiovascular system to augment the pumping action of the heart. The blood pump is sometimes referred to as a mechanical auxiliary ventricle assist device, dynamic aortic patch, balloon pump, mechanical circulatory assist device, or a total mechanical heart. Alternatively, the blood pump can be inserted endovascularly. Typically, the blood pump systems include a driveline that serves as a power and/or signal conduit between the blood pump internal to the patient and a controller/console external to the patient. Additional external medical devices may illustratively include implantable pumps such as insulin pumps and colostomy bags. Such devices are well suited for use with an inventive chain mail collar 302 as shown in FIG. 5 and described above.

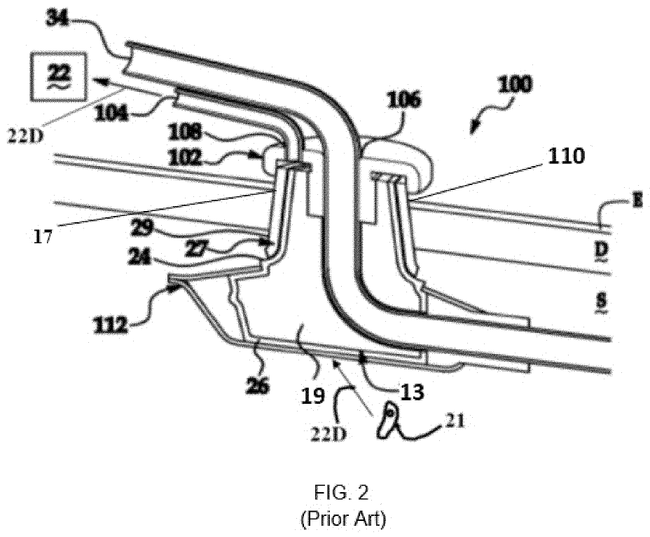

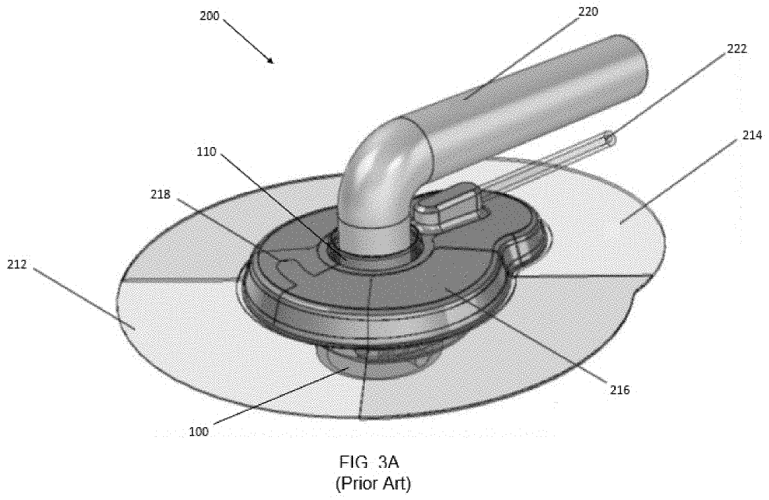

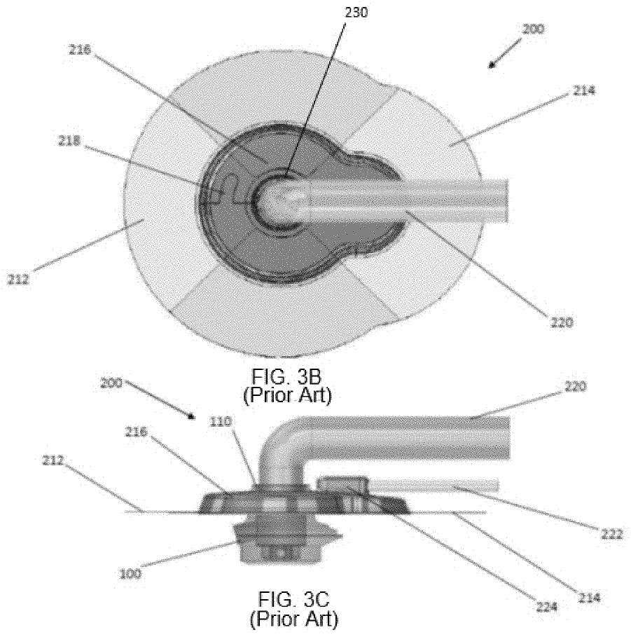

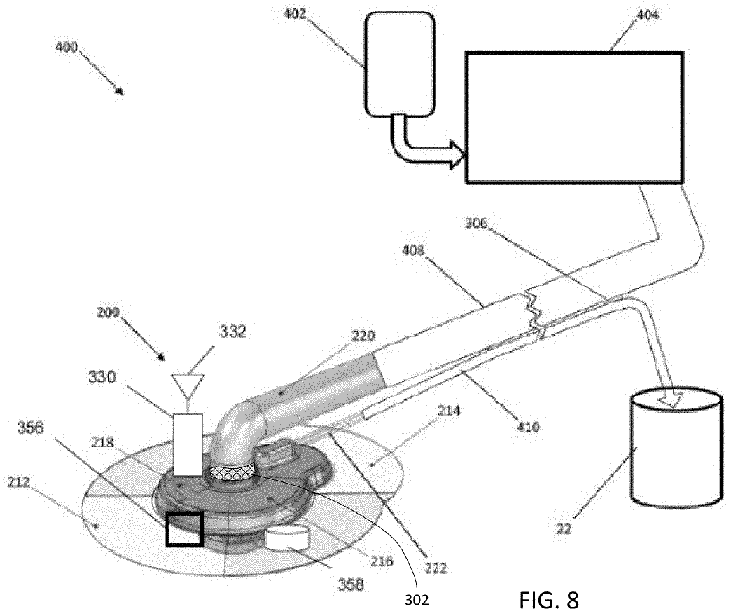

For example, FIG. 8 illustrates a system 400 for suppling intravenous (IV) fluids and a vacuum via an embodiment of the integrated muti-lumen tubes to the modular external interface seal 200 of FIGS. 3A-3C for PAD appliances. According to embodiments, an elongated chain mail collar 302 is attached to a driveline 220 of the PAD 200 at an insertion site. The chain mail collar 302 is percutaneous through the skin line (SL) and the epidermis, dermis, and subcutaneous layers that are denoted at E, D, and S, respectively, and the driveline extends to a vein V, as shown in FIG. 5. An intravenous bag or bottle 402 is shown supplying an infusion pump 404. The IV fluids are supplied via an infusion line 408 to the driveline 220 of the PAD 200. A vacuum line 410 attached to the infusion line 408 with web 306 terminates in a vacuum pump 22 and the vacuum line 222 of the PAD 200. The system 400 further includes sensor 330 that may determine hermaticity with measurements of humidity in the vacuum line 222 to the PAD 200. Alternatively, the sensor 330 may determine the hermaticity of the skin wound in the vicinity of the skin-PAD interface as measured as a function of the fluid exudate or transudate egressing from the skin wound in the vicinity of the skin-PAD interface. Sensor 330 may also measure pressure at the wound site. As shown an observation window 356 allows a healthcare provider assess the condition of the wound without disturbing the vacuum seal. A filter 358 in fluid communication with the wound site provides filtered air that is free of pathogens and aerates the wound.

As noted above, the chain mail collar 302 in some inventive embodiments is adhered to the driveline 220 with an adhesive. In a specific inventive embodiment, the chain mail collar 302 may be attached to the driveline 220 by sutures. In a specific embodiment the chain mail collar 302 may be locally heated to a temperature sufficient to melt an outer surface of the driveline 220 to the chain mail collar 302. In still other inventive embodiments as shown in FIG. 5, a fiber 305 is interwoven into the collar 302. Only a single fiber 305 is shown for visual clarity, it is appreciated that numerous such fibers can be present. The fiber 305 illustratively including monofilament, polyfilament, hollow fibers, or combinations thereof. A fiber 305 modifies properties by affording capillary draw to promote drying, fibroblast infiltration, and in some circumstances monitoring of serous fluid for early indications of infection. Hollow fibers are particularly well suited for such sampling. According to embodiments, the fiber 305 is used in combination with sensor 330 of FIG. 8. The sensor 330 may have leads extending external to the skin such as via a fiber 305 or monitored wirelessly.

Patent documents and publications mentioned in the specification are indicative of the levels of those skilled in the art to which the invention pertains. These documents and publications are incorporated herein by reference to the same extent as if each individual document or publication was specifically and individually incorporated herein by reference.

The foregoing description is illustrative of particular embodiments of the invention, but is not meant to be a limitation upon the practice thereof. The following claims, including all equivalents thereof, are intended to define the scope of the invention.

1. A percutaneous access device (PAD) comprising:
a chain mail collar formed from a plurality of intersecting ringlets formed of biocompatible materials; and
a central aperture through said chain mail collar.

2. The device of claim 1 further comprising a sensor for measuring healing in vivo proximal to said chain mail collar.

3. The device of claim 1 wherein said chain mail collar is formed by three dimensional (3-D) printing.

4. The device of claim 1 further comprising an additional layer of chain mail added to a specific area to increase a diameter of said chain mail collar in the specific area.

5. The device of claim 1 wherein one of said plurality of intersecting ringlets is treated with an anti-microbial substance or a substance that encourages fibroblast attachment and growth thereon.

6. The device of claim 1 further comprising a fiber in contact with or interwoven into one of said plurality of intersecting ringlets.

7. The device of claim 6 wherein said fiber is of the form of a monofilament, a polyfilament, or a hollow fiber.

8. The device of claim 1 further comprising a sensor positioned to monitor a condition of wound healing proximal to said chain mail collar.

9. The device of claim 8 wherein said sensor is at least one of a thermocouple, a gas sensor, an exudate biochemical detector, or a protein detector.

10. The device of claim 8 wherein said sensor communicates data via leads or wireles sly.

11. The device of claim 1 wherein said chain mail collar is joined to a flexible or pliable driveline at an insertion site.

12. The device of claim 11 wherein said chain mail collar is joined to the flexible or pliable driveline with an adhesive.

13. The device of claim 11 wherein said chain mail collar is joined to the flexible or pliable driveline with sutures.

14. The device of claim 11 wherein said chain mail collar is joined to the flexible or pliable driveline by heat treatment to melt an outer surface of the driveline to the collar.

15. The device of claim 1 wherein said collar further comprises a transverse appendage, a distal appendage, or a combination thereof.

16. The device of claim 1 further comprising a vacuum source of hydrostatic source in fluid communication with said chain mail collar.

17. A process of repairing a hernia comprising:
inserting a chain mail sheet formed from a plurality of intersecting ringlets formed of biocompatible materials proximal to weakened or ruptured luminal area of a subject; and
adhering said chain mail sheet as a reinforcement mesh across the weakened or ruptured luminal area.

18. The process of claim 17 wherein said chain mail sheet is collapsed for insertion into the patient and then unfurled at the weakened or ruptured luminal area.

19. The process of claim 18 wherein said chain mail sheet is inserted laparoscopically.

20. The process of claim 17 further comprising creating a vacuum draw or hydrostatic draw through said chain mail sheet.
